(19) United States
(12) Patent Application Publication
(10) Pub. No.: US 2023/0391694 A1
(43) Pub. Date: Dec. 7, 2023

(54) PROCESS FOR THE PRODUCTION OF TRIFLUOROETHYLENE

(71) Applicant: ARKEMA FRANCE, Colombes (FR)

(72) Inventors: Alexandre CAMBRODON, Pierre-Benite Cedex (FR); Thierry LANNUZEL, Pierre-Benite Cedex (FR); Cédric LAVY, Pierre-Benite Cedex (FR); Philippe LEDUC, Pierre-Benite Cedex (FR); Kevin HISLER, Pierre-Benite Cedex (FR)

(73) Assignee: ARKEMA FRANCE, Colombes (FR)

(21) Appl. No.: 18/250,993

(22) PCT Filed: Oct. 27, 2021
(86) PCT No.: PCT/FR2021/051882

§ 371 Date: Apr. 28, 2023

(30) Foreign Application Priority Data
Nov. 3, 2020 (FR) FR2011261

(51) Int. Cl. C07C 17/23; B01J 37/18; B01J 37/08; B01J 23/44

(52) U.S. Cl. CPC C07C 17/23; B01J 23/44; B01J 37/08; B01J 37/18

(57) ABSTRACT

A process for the production of trifluoroethylene in a reactor provided with a catalytic bed including a catalyst, the process including: i) a step of activating the catalyst, including bringing it into contact with a gaseous stream including a reducing agent, an inert gas or a mixture thereof; and ii) a step of reacting chlorotrifluoroethylene with hydrogen in the presence of the catalyst activated in step i) and in the gas phase in order to produce a stream including trifluoroethylene; wherein during said step i), the temperature of the catalytic bed is increased from a temperature T1 to a temperature T2 that is greater than T1, with a temperature gradient of less than 0.5° C./min.

### TECHNICAL FIELD

The present invention relates to a process for the production of hydrofluoroolefins. In particular, the present invention relates to a process for the production of trifluoroethylene (VF₃) by hydrogenolysis of chlorotrifluoroethylene.

### TECHNOLOGICAL BACKGROUND OF THE INVENTION

Fluorinated olefins, such as VF₃, are known and are used as monomers or comonomers for the manufacture of fluorocarbon polymers exhibiting noteworthy characteristics, in particular excellent chemical resistance and good heat resistance.

Trifluoroethylene is a gas under standard conditions of pressure and temperature. The main risks associated with the use of this product relate to its flammability, its propensity for self-polymerization when it is not stabilized, its explosiveness due to its chemical instability and its assumed sensitivity to peroxidation, by analogy with other halogenated olefins. Trifluoroethylene has the distinguishing feature of being extremely flammable, with a lower explosive limit (LEL) of approximately 10% and an upper explosive limit (UEL) of approximately 30%. The main hazard, however, is associated with the propensity of VF₃ to decompose violently and explosively under certain pressure conditions in the presence of an energy source, even in the absence of oxygen.

Given the main risks above, the synthesis and also the storage of VF₃ pose particular problems and impose strict safety rules throughout these processes. A known route for the preparation of trifluoroethylene uses, as starting materials, chlorotrifluoroethylene (CTFE) and hydrogen in the presence of a catalyst and in the gas phase. A process is known from WO2013/128102 for the production of trifluoroethylene by hydrogenolysis of CTFE in the gas phase and in the presence of a catalyst based on a metal from group VIII at atmospheric pressure and at relatively low temperatures.

### SUMMARY OF THE INVENTION

According to a first aspect, the present invention relates to a process for the production of trifluoroethylene in a reactor provided with a catalytic bed comprising a catalyst, said process comprising:
i) a step of activating said catalyst, comprising bringing it into contact with a gaseous stream comprising a reducing agent, an inert gas or a mixture thereof; and
ii) a step of reacting chlorotrifluoroethylene with hydrogen in the presence of the catalyst activated in step i) and in the gas phase in order to produce a stream comprising trifluoroethylene;
   characterized in that, during said step i), the temperature of the catalytic bed is increased from a temperature T1 to a temperature T2 that is greater than T1, with a temperature gradient of less than 0.5° C./min. The temperature gradient applied during the activation makes it possible to prevent premature degradation of the catalyst and thus enables a better yield or better productivity of the hydrogenolysis reaction. It has been observed that the activation of the catalyst with a temperature gradient of greater than 0.5° C./min led to a less efficient process, i.e. poorer trifluoroethylene productivity.

According to a preferred embodiment, the temperature T1 is between 0° C. and 150° C., advantageously between 020 C. and 120° C., preferably between 0° C. and 100° C., more preferentially between 10° C. and 100° C., in particular between 20° C. and 100° C., more particularly between 20° C. and 75° C., favorably between 20° C. and 50° C.

According to the first aspect, the present invention also relates to a process for the production of trifluoroethylene in a reactor provided with a catalytic bed comprising a catalyst, said process comprising:
i) a step of activating said catalyst, comprising bringing it into contact with a gaseous stream comprising a reducing agent, an inert gas or a mixture thereof; and
ii) a step of reacting chlorotrifluoroethylene with hydrogen in the presence of the catalyst activated in step i) and in the gas phase in order to produce a stream comprising trifluoroethylene;
   characterized in that, during said step i), the temperature of the catalytic bed is increased in increments from a temperature T1 to a temperature T2 that is greater than T1.

The present process thus makes it possible to improve the efficiency of the reaction for the hydrogenolysis of chlorotrifluoroethylene to give trifluoroethylene, by virtue of the present step of activating the catalyst that employs heating by consecutive increments. In particular, the productivity of the hydrogenolysis reaction (step ii)) is significantly improved by the activation step i).

According to a preferred embodiment, in step i), between two increments, the temperature is increased with a temperature gradient of less than 0.5° C./min. The temperature gradient applied between two increments makes it possible to prevent premature degradation of the catalyst and thus enables a better yield or better productivity of the hydrogenolysis reaction. According to a preferred embodiment, step i) comprises at least one increment at a temperature T1a of between 90 and 120° C.

According to a preferred embodiment, each increment lasts for between 5 min and 24 h, preferably between 30 min and 10 h.

According to a preferred embodiment, the temperature T2 is between 150° C. and 400° C. According to a particular embodiment, the reducing agent is selected from the group consisting of hydrogen, carbon monoxide, nitric oxide, formaldehyde, C₁-C₆ alkanes and C₁-C₁₀ halohydrocarbons, or a mixture thereof, preferably from hydrogen and C₁-C₁₀ halohydrocarbons or a mixture thereof, in particular from hydrogen, chlorotrifluoroethylene, trifluoroethane, trifluoroethylene, chlorotrifluoroethane or difluoroethane or a mixture thereof. According to the first aspect, the present invention also relates to a process for the production of trifluoroethylene in a reactor provided with a catalytic bed comprising a catalyst, said process comprising:
i) a step of activating said catalyst; and
ii) a step of reacting chlorotrifluoroethylene with hydrogen in the presence of the catalyst activated in step i) and in the gas phase in order to produce a stream comprising trifluoroethylene;
   characterized in that said step i) comprising bringing said catalyst into contact with a gaseous stream comprising chlorotrifluoroethylene and optionally hydrogen. According to a preferred embodiment, during said step i), the temperature of the catalytic bed is increased from a temperature T1′ to a temperature T2′ that is greater than T1′; the temperature of the catalytic bed is preferably increased from a temperature T1′ to a temperature T2′ that is greater than T1′ with a temperature gradient of less than 0.5° C./min. According to a preferred embodiment, the temperature of the catalytic bed is increased by increasing the contact time, calculated as being the ratio of the volume, in liters, of catalyst to the total flow rate of said gaseous stream, in standard liters per second, at the inlet of the reactor.

According to a preferred embodiment, step ii) is carried out at a temperature T3 and the temperature T2′ is less than the temperature T3 at which step ii) is carried out.

According to a second aspect, the present invention relates to a process for the production of trifluoroethylene in a reactor provided with a catalytic bed comprising a catalyst, said process comprising:
a) a step of reacting chlorotrifluoroethylene with hydrogen, carried out in the presence of the catalyst, in the gas phase and at a temperature of the catalytic bed T3, in order to produce a stream comprising trifluoroethylene;
b) a step of regenerating the catalyst used in step a);
c) repeating steps a) and b) using the catalyst regenerated in step b),
   characterized in that step b) is carried out at a temperature of the catalytic bed T4 of from 90° C. to 300° C. and in the presence of hydrogen, preferably at a temperature of the catalytic bed T4 of from 90° C. to 250° C., more preferentially from 90° C. to 200° C., in particular from 90° C. to 175° C., more particularly at a temperature of the catalytic bed T4 of from 90° C. to 150° C. According to a preferred embodiment, the temperature difference AT between the temperature of the catalytic bed in step a) and the temperature of the catalytic bed in step b) [symbol] ΔT=T3−T4, is, in absolute value, between 0 and 50, preferably between 0 and 20.

According to a preferred embodiment, step a) is preceded by a step of activating the catalyst, preferably as described in step i) according to the first aspect of the present invention. According to a preferred embodiment, the process also comprises a step d) of heat treatment of said catalyst at a temperature T5 greater than T4, preferably at a temperature T5 greater than 230° C., in particular at a temperature T5 greater than 250° C. According to a preferred embodiment, said catalyst is a catalyst based on a metal from groups 8 to 10 of the periodic table, preferably deposited on a support, in particular an aluminum-based support.

According to a third aspect, the present invention relates to a process for the production of trifluoroethylene in two reactors each provided with at least one catalytic bed comprising a catalyst, said process comprising:
in a first reactor, a step of reacting chlorotrifluoroethylene with hydrogen, carried out in the presence of the catalyst and in the gas phase in order to produce a stream comprising trifluoroethylene;
in a second reactor, a step of heat treatment of said catalyst. According to a preferred embodiment, said reaction step and said heat treatment step are carried out alternately in each of said two reactors.

According to a preferred embodiment, said heat treatment step is a step of activating the catalyst as described in step i) according to the first aspect of the present invention, or a step of regenerating the catalyst as described in step b) according to the second aspect of the present invention, or a heat treatment according to step d) according to the second aspect of the present invention.

### BRIEF DESCRIPTION OF THE FIGURES

FIG. 1 schematically depicts a device for carrying out the process according to a particular embodiment of the invention wherein the reaction step is carried out in a reactor.

FIG. 2 schematically depicts a device for carrying out the process according to a particular embodiment of the invention wherein the step of activation or regeneration of the catalyst is carried out in a reactor in the presence of nitrogen from a temperature T1 to T1a.

FIG. 2a schematically depicts a device for carrying out the process according to a particular embodiment of the invention wherein the step of activation or regeneration of the catalyst is carried out in a reactor in the presence of hydrogen from a temperature T1a to T2.

FIG. 2b schematically depicts a device for carrying out the process according to a particular embodiment of the invention wherein the step of activation or regeneration of the catalyst is carried out in a reactor in the presence of chrlorotrifluoroethylene from a temperature T1a to T2.

FIG. 3 schematically depicts a device comprising two reactors for carrying out the process, according to a particular embodiment of the invention, wherein the regeneration and reaction steps are alternated, the regeneration step being carried out in the presence of hydrogen.

FIG. 4 schematically depicts a device comprising two reactors for carrying out the process, according to a particular embodiment of the invention, wherein the regeneration and reaction steps are alternated, the regeneration step being carried out in the presence of a halohydrocarbon.

FIG. 5 schematically depicts a device comprising two reactors for carrying out the process, according to another particular embodiment of the invention, wherein the regeneration and reaction steps are alternated.

### DETAILED DESCRIPTION OF THE INVENTION

According to a preferred embodiment, the processes according to the invention described in the present application are carried out continuously.

According to a preferred embodiment, in any one of the processes described in the present application, the hydrogen is in anhydrous form. According to a preferred embodiment, in any one of the processes described in the present application, the chlorotrifluoroethylene is in anhydrous form.

The implementation of the processes according to the invention in the presence of anhydrous hydrogen and/or chlorotrifluoroethylene makes it possible to effectively increase the lifetime of the catalyst and thus the overall productivity of the process. The term anhydrous refers to a content by weight of water of less than 1000 ppm, advantageously 500 ppm, preferably of less than 200 ppm, in particular of less than 100 ppm, based on the total weight of the compound under consideration.

Preferably, the catalyst is based on a metal from columns 8 to 10 of the Periodic Table of the Elements. In particular, the catalyst is based on a metal selected from the group consisting of Pd, Pt, Rh, and Ru; preferably palladium. Preferably, the catalyst is supported. The support is preferably selected from the group consisting of activated carbon, alumina, calcium carbonate and graphite. Preferably, the support is based on aluminum. In particular, the support is alumina.

Thus, the catalyst is more particularly palladium supported on alumina. The alumina can be a-alumina. Preferably, the alumina comprises at least 90% of a-alumina. It was observed that the conversion of the hydrogenolysis reaction was improved when the alumina is an a-alumina.

Preferably, the palladium represents from 0.01% to 5% by weight based on the total weight of the catalyst, preferably from 0.1% to 2% by weight, based on the total weight of the catalyst.

### Activation of the Catalyst (step i))

### Embodiment (1)

According to a first embodiment, the step of activating the catalyst comprises bringing said catalyst into contact with a gaseous stream comprising a reducing agent, an inert gas or a mixture thereof. Preferably, during step i), the temperature of the catalytic bed is increased from a temperature T1 to a temperature T2. In particular, during said step i), the temperature of the catalytic bed is increased from a temperature T1 to a temperature T2 that is greater than T1, with a temperature gradient of less than 0.5° C./min. The temperature gradient applied makes it possible to prevent premature degradation of the catalyst and thus enables a better yield or better productivity of the hydrogenolysis reaction. In particular, the temperature is increased with a temperature gradient of less than 0.45° C./min or less than 0.40° C./min, or less than 0.35° C./min, or less than 0.30° C./min, or less than 0.25° C./min, or less than 0.20° C./min, or less than 0.15° C./min, or less than 0.10° C./min, or less than 0.05° C./min.

The temperature T1 is the initial temperature of the activation step. This temperature T1 can be ambient temperature. Alternatively, the temperature T1 can be between 0° C. and 150° C., advantageously between 0° C. and 120° C., preferably between 0° C. and 100° C., more preferentially between 10° C. and 100° C., in particular between 20° C. and 100° C., more particularly between 20° C. and 75° C., favorably between 20° C. and 50° C.

The temperature T2 is the temperature to be reached during the activation phase. The temperature T2 is advantageously between 150° C. and 400° C., preferably between 155° C. and 375° C., more preferentially between 160° C. and 350° C., in particular between 165° C. and 325° C., more particularly between 170° C. and 320° C., favorably between 175° C. and 310° C., more favorably between 180° C. and 300° C. According to a preferred embodiment, the temperature T2 is advantageously between 185° C. and 290° C., preferably between 190° C. and 280° C., more preferentially between 195° C. and 270° C., in particular between 200° C. and 260° C.

The temperature T2 can be maintained for from 5 min to 200 h, preferably from 10 min to 100 h, in particular from 15 min to 75 h, more particularly from 30 min to 50 h, favorably from 1 h to 25 h. The temperature T2 can be maintained for from 5 min to 24 h, preferably from 10 min to 20 h, in particular from 15 min to 15 h, more particularly from 30 min to 10 h, favorably from 1 h to 10 h.

As mentioned above, the gaseous stream can comprise a reducing agent, an inert gas or a mixture of the two. According to a particular embodiment, the reducing agent is selected from the group consisting of hydrogen, carbon monoxide, nitric oxide, formaldehyde, C₁-C₆ alkanes and C₁-C₁₀ halohydrocarbons, or a mixture thereof, preferably from hydrogen, a C₁-C₁₀ halohydrocarbon or a mixture thereof, in particular from hydrogen, chlorotrifluoroethylene, trifluoroethane, trifluoroethylene, chlorotrifluoroethane or difluoroethane or a mixture thereof. The inert gas may be nitrogen or argon, preferably nitrogen.

The gaseous stream used during step i) preferably does not comprise oxygen. Preferably, step i) can be carried out with an amount of reducing agent of greater than 0.01 mol per gram of catalyst, preferably greater than 0.05 per gram of catalyst. In particular, step i) can be carried out with an amount of reducing agent of between 0.01 and 10 mol per gram of catalyst, preferably between 0.05 and 5 mol per gram of catalyst.

### Embodiment (2)

According to a second embodiment, the step of activating the catalyst comprises bringing said catalyst into contact with a gaseous stream comprising a reducing agent, an inert gas or a mixture thereof. Preferably, during step i), the temperature of the catalytic bed is increased from a temperature T1 to a temperature T2 in increments. The activation of the catalyst in increments makes it possible to increase the performance of the catalyst. Implementing increments makes it possible to prevent degradation of the catalyst. It has also been observed that the properties of the catalyst were additionally further improved if the rise in temperature between the increments is gradual and relatively slow compared to the usual conditions for activating a catalyst.

Thus, preferably, in step i), between two increments, the temperature is increased with a temperature gradient of less than 0.5° C./min. The temperature gradient applied between two increments makes it possible to prevent premature degradation of the catalyst and thus enables a better yield or better productivity of the hydrogenolysis reaction. In particular, the temperature is increased with a temperature gradient of less than 0.45° C./min or less than 0.40° C./min, or less than 0.35° C./min, or less than 0.30° C./min, or less than 0.25° C./min, or less than 0.20° C./min, or less than 0.15° C./min, or less than 0.10° C./min, or less than 0.05° C./min. The temperature T1 is the initial temperature of the activation step. This temperature T1 can be ambient temperature. Alternatively, the temperature T1 can be between 0° C. and 150° C., advantageously between 0° C. and 120° C., preferably between 0° C. and 100° C., more preferentially between 10° C. and 100° C., in particular between 20° C. and 100° C., more particularly between 20° C. and 75° C., favorably between 20° C. and 50° C. The temperature T2 is the temperature to be reached during the activation phase. The temperature T2 is advantageously between 150° C. and 400° C., preferably between 155° C. and 375° C., more preferentially between 160° C. and 350° C., in particular between 165° C. and 325° C., more particularly between 170° C. and 320° C., favorably between 175° C. and 310° C., more favorably between 180° C. and 300° C. According to a preferred embodiment, the temperature T2 is advantageously between 185° C. and 290° C., preferably between 190° C. and 280° C., more preferentially between 195° C. and 270° C., in particular between 200° C. and 260° C. The temperature T2 can be maintained for from 5 min to 200 h, preferably from 10 min to 100 h, in particular from 15 min to 75 h, more particularly from 30 min to 50 h, favorably from 1 h to 25 h. The temperature T2 can be maintained for from 5 min to 24 h, preferably from 10 min to 20 h, in particular from 15 min to 15 h, more particularly from 30 min to 10 h, favorably from 1 h to 10 h.

Step i) of activating the catalyst contains at least one increment between the temperature T1 and the temperature T2. Step i) of activating the catalyst can comprise several increments between the temperature T1 and the temperature T2. Preferably, step i) comprises at least one increment at a temperature T1a of between 90 and 120° C. The presence of an increment between 90° C. and 120° C. should be favored for increasing the lifetime of the catalyst. Step i) can also comprise one or more increments between the temperature T1 and T1a and/or between the temperature T1a and T2.

Preferably, each increment between the temperature T1 and the temperature T2 can last between 5 min and 200 h, preferably between 10 min and 100 h, in particular between 15 min and 75 h, more particularly between 30 min and 50 h. In particular, each increment between the temperature T1 and the temperature T2 can last between 5 min and 24 h, preferably between min and 20 h, in particular between 15 min and 15 h, more particularly between 30 min and h. In particular, the increment at the temperature T1a can last between 5 min and 200 h, preferably between 10 min and 100 h, in particular between 15 min and 75 h, more particularly between 30 min and 50 h. Favorably, the increment at the temperature T1a can last between 5 min and 24 h, preferably between 10 min and 20 h, in particular between 15 min and 15 h, more particularly between 30 min and 10 h.

As mentioned above, the gaseous stream can comprise a reducing agent, an inert gas or a mixture of the two. According to a particular embodiment, the reducing agent is selected from the group consisting of hydrogen, carbon monoxide, nitric oxide, formaldehyde, C₁-C₆ alkanes and C₁-C₁₀ halohydrocarbons, or a mixture thereof, preferably from hydrogen or a C₁-C₁₀ halohydrocarbon or a mixture thereof, in particular from hydrogen, chlorotrifluoroethylene, trifluoroethane, trifluoroethylene, chlorotrifluoroethane or difluoroethane or a mixture thereof.

The inert gas may be nitrogen or argon, preferably nitrogen.

The gaseous stream used during step i) preferably does not comprise oxygen.

The gaseous stream used during step i) can be different over time. For example, the gaseous stream can comprise an inert gas between two increments and for example comprise a reducing agent between two other increments. In particular, the gaseous stream comprises an inert gas when step i) is carried out between the temperature T1 and T1a, and the gaseous stream comprises a reducing agent, preferably hydrogen or C₁-C₁₀ halohydrocarbons as defined above, when step i) is carried out between the temperature T1a and T2. Thus, the gaseous stream used during step i) is modified during the increment carried out at the temperature T1a.

Alternatively, the gaseous stream can comprise a reducing agent such as hydrogen or C₁-C₁₀ halohydrocarbons as defined above throughout the whole of step i), optionally mixed with an inert gas such as nitrogen. It has been observed that the use of a reducing agent such as hydrogen or C₁-C₁₀ halohydrocarbons as defined above, optionally mixed with an inert gas such as nitrogen, during the temperature rise between the temperature T1a of said increment and the temperature T2, represents an additional advantage in terms of productivity. As mentioned above, the temperature T2 is maintained for a certain period of time. During this increment at the temperature T2, the gaseous stream can be modified. Thus, the gaseous stream, during the increment at the temperature T2, can comprise hydrogen or a C₁-C₁₀ halohydrocarbon as defined above; in particular, the gaseous stream, during the increment at the temperature T2, can comprise hydrogen, chlorotrifluoroethylene, trifluoroethane, trifluoroethylene, chlorotrifluoroethane or difluoroethane.

Preferably, step i) can be carried out with an amount of reducing agent of greater than 0.01 per gram of catalyst, preferably greater than 0.05 per gram of catalyst. In particular, step i) can be carried out with an amount of reducing agent of between 0.01 and 10 mol per gram of catalyst, preferably between 0.05 and 5 mol per gram of catalyst.

### Embodiment (3)

According to a third embodiment, step i) of activating the catalyst comprises bringing said catalyst into contact with a gaseous stream that comprises chlorotrifluoroethylene and optionally hydrogen. It has been observed that chlorotrifluoroethylene (CTFE) made it possible to activate the catalyst, in particular when only hydrogen is also present. This makes it possible to improve the process for the production of trifluoroethylene. Activation in the presence of CTFE makes it possible to activate the catalyst at lower temperatures and thus provides a less energy-consuming process. The process is further simplified because the reducing agent during the activation is also one of the reactants for the subsequent reaction.

Preferably, in this embodiment, step i) is carried out at a temperature T2′ of less than 100° C. This temperature T2′ can be reached from a temperature T1′ using a low temperature gradient. Thus, during said step i), the temperature of the catalytic bed is increased from a temperature T1′ to a temperature T2′ that is greater than T1′; the temperature of the catalytic bed is preferably increased from a temperature T1′ to a temperature T2′ that is greater than T1′ with a temperature gradient of less than 0.5° C./min. The temperature gradient applied makes it possible to prevent premature degradation of the catalyst and thus enables a better yield or better productivity of the hydrogenolysis reaction. In particular, the temperature is increased with a temperature gradient of less than 0.45° C./min or less than 0.40° C./min, or less than or less than 0.30° C./min, or less than 0.25° C./min, or less than 0.20° C./min, or less than 0.15° C./min, or less than 0.10° C./min, or less than 0.05° C./min. Preferably, the temperature of the catalytic bed is increased by increasing the contact time, calculated as being the ratio of the volume, in liters, of catalyst to the total flow rate of said gaseous stream, in standard liters per second, at the inlet of the reactor. The contact time is between 1 and 60 seconds, preferably between 5 and 45 seconds, in particular between 10 and seconds, more particularly between 15 and 25 seconds.

The temperature T1′ can be between 0° C. and 50° C., advantageously between 10° C. and 50° C., preferably between 20° C. and 50° C.

Preferably, the temperature T2′ is less than the temperature T3 at which step ii) is carried out. The temperature T3 is preferably between 100° C. and 180° C., more preferentially between 100° C. and 160° C., in particular between 120° C. and 160° C.

### Hydrogenolysis Reaction (step ii) According to the First Aspect of the Invention or Step a) According to the Second Aspect of the Invention)

The Present Invention Comprises, as Mentioned Above, a Step of a Hydrogenolysis Reaction of chlorotrifluoroethylene (CTFE) with hydrogen (step ii) or step a)). The hydrogenolysis step is carried out in the presence of a catalyst and in the gas phase. The hydrogenolysis step is preferably carried out in the presence of said activated catalyst and in the gas phase.

The hydrogenolysis step consists in simultaneously introducing hydrogen, CTFE and optionally an inert gas, such as nitrogen, in the gas phase and in the presence of said catalyst, preferably activated catalyst. The temperature of the catalytic bed, T3, is controlled by circulation of a heat-transfer fluid in the jacket of the reactor. The temperature of the heat-transfer fluid is adapted depending on the targeted temperature T3.

The temperature T3 is preferably between 100° C. and 180° C., more preferentially between 100° C. and 160° C., in particular between 120° C. and 160° C.

The H₂ S/CTFE molar ratio is between 0.5/1 and 2/1, and preferably between 1/1 and 1.2/1. If an inert gas, such as nitrogen, is present in step ii), the nitrogen/H 2 molar ratio is between 0/1 and 2/1 and preferably between 0/1 and 1/1.

Step ii) is preferably carried out at a pressure of 0.05 MPa to 0.2 MPa, in particular at atmospheric pressure.

The contact time, calculated as being the ratio of the volume, in liters, of catalyst to the total flow rate of the gas mixture, in standard liters per second, at the inlet of the reactor, is of between 1 and 60 seconds, preferably between 5 and 45 seconds, in particular between 10 and 30 seconds, more particularly between 15 and 25 seconds.

The hydrogenolysis step of the present process results in the production of a stream A comprising trifluoroethylene. Said stream A can also comprise unreacted hydrogen and unreacted CTFE. The stream A can also comprise trifluoroethane and/or chlorotrifluoroethane as byproducts of the hydrogenolysis reaction.

The stream A can also comprise HCl and HF. Preferably, the stream A is recovered at the reactor outlet in the gaseous form. Preferably, at the outlet of the hydrogenolysis reactor, the stream A is treated in order to remove HCl and HF. The stream A is passed through water in a scrubbing column followed by scrubbing with a dilute base, such as NaOH or KOH. The remainder of the gas mixture, consisting of the unconverted reactants (H₂ and CTFE), dilution nitrogen (if present) and reaction products (VF₃, 143, 133 and other organic products) which form the gas mixture B, is directed to a dryer in order to remove the traces of scrubbing water. Drying can be carried out using products such as calcium, sodium or magnesium sulfate, calcium chloride, potassium carbonate, silica gel or zeolites. In one embodiment, a molecular sieve (zeolite), such as siliporite, is used for the drying. The stream B, thus dried, is subjected to a step of separation of the hydrogen and inert substances from the remainder of the other products present in the mixture B, by absorption/desorption in the presence of an alcohol comprising from 1 to 4 carbon atoms and preferably ethanol, at atmospheric pressure and at a temperature below ambient temperature, preferably of less than 10° C. and even more preferably at a temperature of −25° C., for the absorption. In one embodiment, the absorption of the organic substances is carried out in a countercurrent column with ethanol cooled to −25° C. The ethanol flow rate is adjusted according to the flow rate of organic substances to be absorbed. The hydrogen and inert gases, which are insoluble in ethanol at this temperature, are removed at the absorption column top. The organic substances are subsequently recovered in the form of a gas mixture C, by heating the ethanol to its boiling point (desorption), in order to be subsequently distilled.

Pure trifluoroethylene (VF₃) is subsequently distilled from the mixture C, to be separated from the other organic products (CTFE, F143, F133 and other organic substances, forming a mixture D). The mixture D comprising the other organic compounds is recovered at the column bottom. The distillation of said mixture D on a second column makes it possible to recover and recycle the unconverted CTFE at the column top and to remove the byproducts of the reaction at the bottom of this second column.

### Regeneration of the Catalyst (step b))

Preferably, the regeneration in step b) is carried out at a temperature of the catalytic bed T4 of from 90° C. to 300° C. and in the presence of hydrogen, preferably at a temperature of the catalytic bed T4 of from 90° C. to 250° C., more preferentially from 90° C. to 200° C., in particular from 90° C. to 175° C., more particularly at a temperature of the catalytic bed T4 of from 90° C. to 150° C.

Carrying out step b) makes it possible to improve the reaction yield compared to the initial yield before regeneration. In particular, carrying out step b) at a low temperature, for example from to 200° C. or from 90° C. to 175° C. or from 90° C. to 150° C., enables the desorption of compounds detrimental to the activity of the catalyst and/or makes it possible to limit phase transitions that modify the structure of the catalyst.

According to a preferred embodiment, the temperature difference ΔT between the temperature of the catalytic bed in step a) and the temperature of the catalytic bed in step b) [symbol] MAT=T3−T4, is, in absolute value, between 0 and 50. Advantageously, the temperature difference AT is between 0 and 45, preferably between 0 and 40, in particular between 0 and 35, more particularly between 0 and 30, favorably between 0 and 25, advantageously favorably between and 20, preferentially favorably between 0 and 15, particularly favorably between 0 and 10. The present process makes it possible to carry out both the hydrogenolysis reaction and the catalyst regeneration at low temperature. This represents a significant energy saving. As mentioned above, the low-temperature regeneration makes it possible to improve the initial yield of step a), and not only to match the initial yield.

Preferably, step b) can be carried out for a period of time of from 1 h to 500 h, preferably from 1 h to 400 h, in particular from 1 h to 300 h, more particularly from 1 h to 200 h.

As mentioned above, step b) is carried out in the presence of hydrogen. Step b) of regenerating the catalyst is preferably carried out in the absence of oxidizing agent, more particularly in the absence of a stream containing oxygen. Oxygen as mentioned here refers to pure oxygen or air or to a mixture containing oxygen and nitrogen. Indeed, the presence of an oxidizing agent during step b) may cause the structure of the catalyst to be modified, which would require carrying out step a) at higher temperature. Thus, the present step b) can consist of a regeneration of the catalyst in the presence of a stream consisting of hydrogen, optionally mixed with an inert gas. The present step b) can consist of a regeneration of the catalyst in a single step in the presence of a stream of hydrogen.

### Heat Treatment (Step d))

According to a preferred embodiment, the process also comprises a step d) of heat treatment of said catalyst at a temperature T5 greater than T4, preferably at a temperature T5 greater than 200° C., advantageously greater than 230° C., preferably greater than 250° C., in particular greater than 300° C. Step d) can be carried out periodically depending on the productivity or conversion obtained in step a). Preferably, step d) follows step a), after several repetitions of steps a), b) and c). After carrying out step d), steps a), b) and c) are repeated.

Step d) preferably consists of bringing said catalyst into contact with a gaseous stream comprising a reducing agent, an inert gas or a mixture of the two. The reducing agent is as defined above in the first aspect of the present invention.

Step d) can be carried out by increasing the temperature of the catalytic bed in increments, as described for step i) according to the present invention. Thus, step d) can be carried out starting from a temperature T6 to reach at least one increment at a temperature T6a that is greater than T6, then increasing the temperature T6a to reach the temperature T5.

Preferably, in step d), the temperature of the catalytic bed is increased with a temperature gradient of less than 0.5° C./min. In particular, the temperature is increased with a temperature gradient of less than 0.45° C./min or less than 0.40° C./min, or less than 0.35° C./min, or less than or less than 0.25° C./min, or less than 0.20° C./min, or less than 0.15° C./min, or less than 0.10° C./min, or less than 0.05° C./min.

The temperature T5 is advantageously between 200° C. and 300° C., preferably between 205° C. and 295° C., more preferentially between 210° C. and 290° C., in particular between 215° C. and 290° C., more particularly between 220° C. and 285° C., favorably between 225° C. and 280° C., more favorably between 230° C. and 280° C. Alternatively, the temperature T5 can be between 300° C. and 450° C., preferably between 300° C. and 400° C.

The temperature T5 can be maintained for from 5 min to 200 h, preferably from 10 min to 100 h, in particular from 15 min to 75 h, more particularly from 30 min to 50 h, favorably from 1 h to 25 h.

Preferably, in step d), the temperature T6 is generally ambient temperature. Alternatively, the temperature T6 can be between ambient temperature and 50° C.

Step d) preferably comprises at least one increment at a temperature T6a of between 90 and 120° C.

Preferably, each increment between the temperature T6 and the temperature T5 lasts between min and 200 h, preferably between 10 min and 100 h, in particular between 15 min and 75 h, more particularly between 30 min and 50 h.

Prior to step d), the present process can comprise a step of cooling the catalytic bed. Thus, the catalytic temperature can be decreased by the temperature T3, the temperature of the catalytic bed during step a) at the temperature T6, the initial temperature of the heat treatment of step d).

As mentioned above, the gaseous stream can comprise a reducing agent, an inert gas or a mixture of the two. According to a particular embodiment, the reducing agent is selected from the group consisting of hydrogen, carbon monoxide, nitric oxide, formaldehyde, C₁-C₆ alkanes and C₁-C₁₀ halohydrocarbons, or a mixture thereof, preferably from hydrogen or a C₁-C₁₀ halohydrocarbon or a mixture thereof, in particular from hydrogen, chlorotrifluoroethylene, trifluoroethane, trifluoroethylene, chlorotrifluoroethane or difluoroethane or a mixture thereof.

The inert gas may be nitrogen or argon, preferably nitrogen.

Preferably, the gaseous stream does not comprise oxygen.

Preferably, the gaseous stream used in step d) is the same as that used in step i).

For example, the gaseous stream can comprise an inert gas between two increments and for example comprise a reducing agent between two other increments. In particular, the gaseous stream comprises an inert gas when step d) is carried out between the temperature T6 and T6a, and the gaseous stream comprises a reducing agent, preferably hydrogen or C1-C₁₀ halohydrocarbons as defined above, when step d) is carried out between the temperature T6a and T5. Thus, the gaseous stream used during step d) is modified during the increment carried out at the temperature T6a. Alternatively, the gaseous stream can comprise a reducing agent such as hydrogen or C₁-C₁₀ halohydrocarbons as defined above throughout the whole of step d), optionally mixed with an inert gas such as nitrogen. It has been observed that the use of a reducing agent such as hydrogen or C₁-C₁₀ halohydrocarbons as defined above during the temperature rise between the temperature T6a of said increment and the temperature T5, represents a significant advantage in terms of productivity.

Preferably, step d) can be carried out with an amount of reducing agent of greater than 0.01 per gram of catalyst, preferably greater than 0.05 per gram of catalyst. In particular, step d) can be carried out with an amount of reducing agent of between 0.01 and 10 mol per gram of catalyst, preferably between 0.05 and 5 mol per gram of catalyst.

### First and Second Aspects of the Invention

As mentioned above, according to a first aspect, the present invention relates to a process for the production of trifluoroethylene. Said process is carried out in a reactor provided with a catalytic bed comprising a catalyst. The process comprises a step of activating the catalyst (step i)) according to any one of embodiments (1) to (3)) and a step of reacting chlorotrifluoroethylene with hydrogen in the presence of the catalyst, preferably activated catalyst, and in the gas phase in order to produce a stream comprising trifluoroethylene (step b)).

As mentioned above, according to a second aspect, the present invention relates to a process for the production of trifluoroethylene in a reactor provided with a catalytic bed comprising a catalyst, said process comprising:
a) a step of reacting chlorotrifluoroethylene with hydrogen, carried out in the presence of the catalyst, in the gas phase and at a temperature of the catalytic bed T3, in order to produce a stream comprising trifluoroethylene;
b) a step of regenerating the catalyst used in step a);
c) repeating steps a) and b) using the catalyst regenerated in step b), Preferably, in this aspect of the invention, the regeneration in step b) is carried out at a temperature of the catalytic bed T4 of from 90° C. to 300° C. and in the presence of hydrogen.

According to a preferred embodiment, step a) is preceded by a step of activating the catalyst, preferably as described in step i) above according to any one of embodiments (1) to (3).

Thus, preferably, the present invention provides a process for the production of trifluoroethylene in a reactor provided with a catalytic bed comprising a catalyst, said process comprising:
a step of activating said catalyst, preferably according to any one of embodiments (1) to (3) of step i);
a step of reacting chlorotrifluoroethylene with hydrogen in the presence of the activated catalyst and in the gas phase in order to produce a stream comprising trifluoroethylene (step ii) or a));
a step of regenerating the catalyst, preferably at a temperature of the catalytic bed T4 of from 90° C. to 300° C. and in the presence of hydrogen (step b));
repeating the reaction and regeneration steps using the regenerated catalyst (step c));
optionally, a step of heat treatment of the catalyst (step d)).

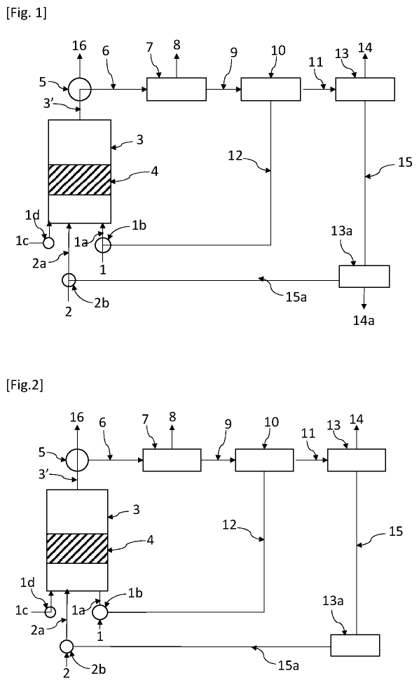

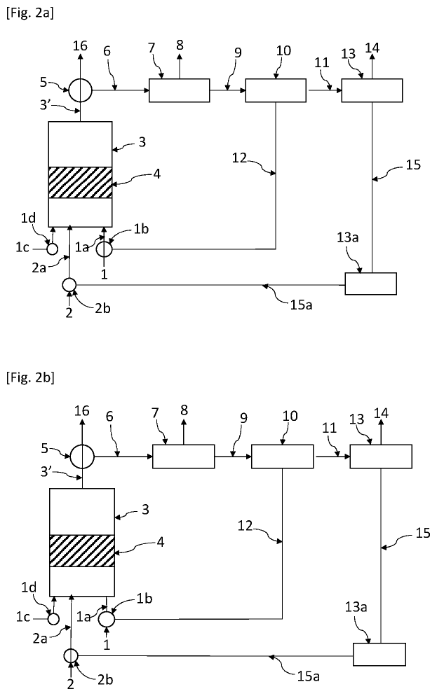

FIGS. 1, 2, 2a and 2b illustrate particular embodiments of the present invention according to the first aspect or the second aspect of the present invention.

FIG. 1 schematically depicts a device for carrying out a process according to the present invention during a hydrogenolysis reaction step. The reactor 3 comprises a catalytic bed 4 and is fed with chlorotrifluoroethylene 2 and with hydrogen 1 via valves 2b and 1b and pipes la and 2a. In one embodiment in which the reaction between hydrogen and CTFE is carried out (step ii) of the first aspect of the invention or step a) of the second aspect of the invention), the valves 1b and 2b are configured to enable the reactor 3 to be fed with CTFE 2 and hydrogen 1. The gases resulting from the hydrogenolysis reaction (stream A) are evacuated from the reactor 3 by the pipe 3′. The valve 5 feeds, via the pipe 6, a device 7 for treating the stream A in order to eliminate acids such as HF or HCl. The treatment device 7 may be a scrubbing column containing water or sodium hydroxide or a combination of the two (firstly scrubbing using water then scrubbing with an alkaline solution such as sodium hydroxide), which makes it possible to limit salt formation. The residues of this aqueous or basic treatment are eliminated at 8. The stream B comprising the remainder of the gas mixture feeds, via the pipe 9, a first purification device with the aim of separating hydrogen and inert gases from the organic compounds present in the stream B. The stream B can be dried over a molecular sieve prior to the separation. The purification device 10 implements an absorption/desorption step in the presence of ethanol at atmospheric pressure and at a temperature below ambient temperature. The hydrogen and inert gases (if present) are recycled via the pipe 12 to the valve 1b and the reactor 3. The organic substances are subsequently recovered in the form of a gas mixture C, by heating the ethanol to its boiling point (desorption). The stream C feeds a second purification device 13 via the pipe 11. The device 13 is preferably a distillation column. The trifluoroethylene is recovered at the top of the distillation column 14. The other organic compounds, including CTFE, are recovered at the bottom of the distillation column and form the mixture D. The latter feeds, via the pipe a third purification device 13a, preferably a distillation column, in order to recover the CTFE which is recycled to the valve 2b and the reactor 3 by way of the pipe 15a. The other organic compounds separated from the CTFE are sent for example to an incinerator or a device for recovering impurities 14a.

FIG. 2 depicts a device for carrying out the present process during a phase of activation or regeneration of the catalyst. The reactor 3 comprises a catalytic bed 4. In this particular embodiment, the catalytic bed is fed with an inert gas 1c (such as nitrogen) via the valve 1d. The gases resulting from the regeneration or activation are evacuated from the reactor 3 via the pipe 3′. The valve 5 transfers the regeneration or activation gases to an incinerator or a device for recovering same 16. The regeneration or activation gases may optionally be recycled after purification (scrubbing with water and with an alkaline solution such as sodium hydroxide) and can be dried over a molecular sieve. For example, in the case of an activation step, the temperature of the catalytic bed 4 can increase from a temperature T1 to T1a. While the increment T1a is maintained, the valve 1d is closed and the valve 1b is opened in order to feed the reactor with hydrogen 1. The temperature of the catalytic bed 4 is then increased from a temperature T1a to T2. This is depicted in FIG. 2a. Alternatively, while the increment T1a is maintained, the valve 1d is closed and the valve 2b is opened in order to feed the reactor with chlorotrifluoroethylene. The temperature of the catalytic bed 4 is then increased from a temperature T1a to T2. This is depicted in FIG. 2b.

### Third Aspect of the Invention

According to a third aspect, the present invention relates to a process for the production of trifluoroethylene in two reactors each provided with at least one catalytic bed comprising a catalyst, said process comprising:
in a first reactor, a step of reacting chlorotrifluoroethylene with hydrogen, carried out in the presence of the catalyst and in the gas phase in order to produce a stream comprising trifluoroethylene;
in a second reactor, a step of heat treatment of said catalyst.

According to a preferred embodiment, said reaction step and said heat treatment step are carried out alternately in each of said two reactors.

According to a preferred embodiment, said heat treatment step is a step of activating the catalyst as described in step i) according to the first aspect of the present invention, or a step of regenerating the catalyst as described in step b) according to the second aspect of the present invention, or a heat treatment as described in step d) according to the second aspect of the present invention.

The step of reacting chlorotrifluoroethylene with hydrogen in this third aspect of the invention is equivalent to the reaction step described in step ii) of the first aspect of the invention or in step a) according to the second aspect of the invention.

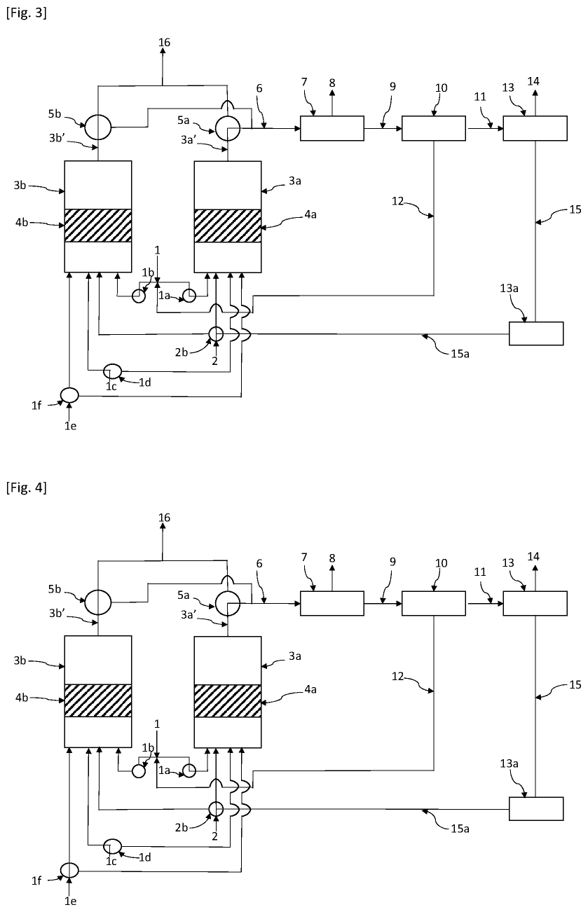

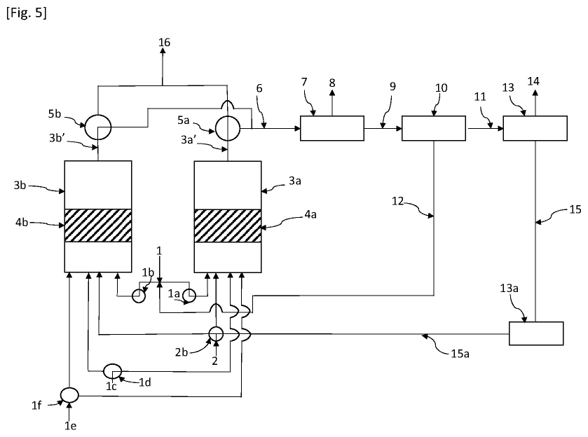

FIG. 3 schematically depicts a device comprising two reactors for carrying out a process according to the third aspect of the invention. The device comprises two reactors 3a and 3b, each comprising a catalytic bed 4a and 4b containing a catalyst. In the embodiment depicted, the reactor 3a is in production mode, i.e. the reactor is carrying out the reaction for the hydrogenolysis of CTFE by hydrogen. The reactor 3b is in heat treatment mode, i.e. the reactor is carrying out a step of activating the catalyst as described in step i) according to the first aspect of the present invention, or a step of regenerating the catalyst as described in step b) according to the second aspect of the present invention, or a heat treatment as described in step d) according to the second aspect of the present invention. The reactor 3a comprises a catalytic bed 4a and is fed with chlorotrifluoroethylene 2 and with hydrogen 1 via valves 2b and 1a. The valves 1a and 2b are configured to enable the reactor 3a to be fed with CTFE 2 and hydrogen 1. The gases resulting from the hydrogenolysis reaction (stream A) are evacuated from the reactor 3a by the pipe 3a′. The valve 5a feeds, via the pipe 6, device 7 for treating the stream A in order to eliminate acids such as HF or HCl. The treatment device 7 can be a scrubbing column containing water or sodium hydroxide. The residues of this aqueous or basic treatment are eliminated at 8. The stream B comprising the remainder of the gas mixture feeds, via the pipe 9, a first purification device 10 with the aim of separating hydrogen and inert gases from the organic compounds present in the stream B. The stream B can be dried over a molecular sieve prior to the separation. The purification device 10 implements an absorption/desorption step in the presence of ethanol at atmospheric pressure and at a temperature below ambient temperature. The hydrogen and inert gases are recycled via the pipe 12 to the valve 1a and the reactor 3a. The organic substances are subsequently recovered in the form of a gas mixture C, by heating the ethanol to its boiling point (desorption). The stream C feeds a second purification device 13 via the pipe 11. The device 13 is preferably a distillation column. The trifluoroethylene is recovered at the top of the distillation column 14. The other organic compounds, including CTFE, are recovered at the bottom of the distillation column and form the mixture D. The latter feeds, via the pipe 15, a third purification device 13a, preferably a distillation column, in order to recover the CTFE which is recycled to the valve 2b and the reactor 3a by way of the pipe 15a. The other organic compounds separated from the CTFE are sent for example to the incinerator. The reactor 3b is fed with hydrogen 1 via the valve 1b and/or with an inert gas 1c (for example nitrogen) via the valve 1d. The feeding with hydrogen 1 or inert gas 1c is easily regulated by the valves 1b and 1d in order to enable the activation or regeneration steps to be carried out. The gaseous stream resulting from the heat treatment of the catalyst carried out in the reactor 3b is recovered via the pipe 3b′ and sent, via the valve 5b, to an incinerator or a recovery device 16. If the heat treatment carried out is an activation according to step i) or according to a treatment according to step d), the reducing agent can for example be a halohydrocarbon as defined in the present application. In this case, the reactor 3b is fed with reducing agent 1e via the valve 1f. This is depicted in FIG. 4. In this case, the valve 1b is configured not to feed the reactor 3b with hydrogen (FIG. 4). Alternatively, if the halohydrocarbn is CTFE, this can feed the reactor 3b via the valve 2b.

FIG. 5 depicts an embodiment in which the reactor 3b is in production mode, i.e. is carrying out the reaction for the hydrogenolysis of CTFE with hydrogen in the gas phase and in the presence of a catalyst, as described in the present application. The reactor 3b is thus fed with CTFE 2 via the valve 2b and with hydrogen via the valve 1b. In this case, the reactor 3a is in catalyst heat treatment mode. In this particular embodiment, the reactor 3a is fed with hydrogen via the valve 1a and with an inert gas 1c via the valve 1d. The feed of each of the gases is regulated over time if necessary, as explained in the present application. Thus, at the outlet of the reactor 3b, the reaction gases (i.e. stream A) are sent to the device 7 via the pipe 3b′ and the pipe 6. The valve 5b is configured for this purpose. The stream A is treated as described above. At the outlet of the reactor 3a, the gases are sent to the incinerator or a recover device 16 via the valve 5a configured for this purpose.

### EXAMPLES

### Example 1

25 cm³ of catalyst (0.2% of palladium supported on α-alumina) are introduced into a tubular reactor consisting of a stainless steel tube with a length of 1200 mm over a diameter of 25 mm, and equipped with a jacket. The catalyst, thus charged, was subsequently activated in the following way: the reaction tube was placed in a tube furnace and was fed with a stream of hydrogen (from 0.05 to 0.1 mol per gram of catalyst). The catalytic bed was then heated to a temperature of 200° C. to 250° C. with a temperature gradient of 0.2° C./min. After this activation period, the tube was cooled to ambient temperature and then was isolated in order to then be installed on a hydrogenolysis test bench. The reactor was fed with 1 mol/h of CTFE and 1 mol/h of hydrogen. It is also possible to feed the reactors with an inert gas (in this instance nitrogen). The temperature of the catalytic bed was between 100° C. and 130° C. The contact time, calculated as being the ratio of the volume in liters of catalyst to the sum of the flow rates of the reactants in standard liters per second, was approximately 22 seconds. After reacting and purifying the gases obtained at the outlet of the reactor, the trifluoroethylene productivity was 18 g/h.

### Example 2 (Comparative)

Example 1 was reproduced with a temperature gradient of 0.7° C./min. After reacting and purifying the gases obtained at the outlet of the reactor, the trifluoroethylene productivity was less than 7.5 g/h.

### Example 3: Regeneration According to the Invention

The device of example 1 is used in this example. The catalyst was activated as in example 1. The reactor was fed with 1 mol/h of CTFE and 1 mol/h of hydrogen. It is also possible to feed the reactors with an inert gas (in this instance nitrogen). The temperature of the catalytic bed was between 100° C. and 130° C. The contact time, calculated as being the ratio of the volume in liters of catalyst to the sum of the flow rates of the reactants in standard liters per second, was approximately 22 seconds. The productivity was approximately 18.5 g/h. After 100 h, the catalyst was regenerated under a stream of hydrogen (stoppage of the CTFE stream) at a temperature of between 90° C. and 150° C. for 48 h. The reactor was then fed with 1 mol/h of CTFE and 1 mol/h of hydrogen. After reacting and purifying the gases obtained at the outlet of the reactor, the trifluoroethylene productivity was 25 g/h.

1. A process for the production of trifluoroethylene in a reactor provided with a catalytic bed comprising a catalyst, said process comprising:
i) a step of activating said catalyst, comprising bringing it into contact with a gaseous stream comprising a reducing agent, an inert gas or a mixture thereof; and
ii) a step of reacting chlorotrifluoroethylene with hydrogen in the presence of the catalyst activated in step i) and in the gas phase in order to produce a stream comprising trifluoroethylene;
wherein during said step i), the temperature of the catalytic bed is increased from a temperature T1 to a temperature T2 that is greater than T1, with a temperature gradient of less than 0.5° C./min.

2. The process as claimed in claim 1, wherein the temperature T1 is between 0° C. and 150°C.

3. A process for the production of trifluoroethylene in a reactor provided with a catalytic bed comprising a catalyst, said process comprising:
i) a step of activating said catalyst, comprising bringing it into contact with a gaseous stream comprising a reducing agent, an inert gas or a mixture thereof; and
ii) a step of reacting chlorotrifluoroethylene with hydrogen in the presence of the catalyst activated in step i) and in the gas phase in order to produce a stream comprising trifluoroethylene;
wherein during said step i), the temperature of the catalytic bed is increased in increments from a temperature Tito a temperature T2 that is greater than T1.

4. The process as claimed in claim 3, wherein step i), between two increments, the temperature is increased with a temperature gradient of less than 0.5° C./min.

5. The process as claimed claim 3, wherein step i) comprises at least one increment at a temperature T1a of between 90 and 120° C.

6. The process as claimed claim 3, wherein each increment lasts for between 5 min and 24 h.

7. The process as claimed claim 1, where in the temperature T2 is between 150° C. and 400° C.

8. The process as claimed claim 1, wherein the reducing agent is selected from the group consisting of hydrogen, carbon monoxide, nitric oxide, formaldehyde, C1-C6 alkanes and C1-C10 halohydrocarbons, or a mixture thereof.

9. A process for the production of trifluoroethylene in a reactor provided with a catalytic bed comprising a catalyst, said process comprising:
i) a step of activating said catalyst; and
ii) a step of reacting chlorotrifluoroethylene with hydrogen in the presence of the catalyst activated in step i) and in the gas phase in order to produce a stream comprising trifluoroethylene;
wherein step i) comprising said catalyst into contact with a gaseous stream comprising chlorotrifluoroethylene and optionally hydrogen.

10. The process as claimed in claim 9, wherein during said step i), the temperature of the catalytic bed is increased from a temperature T1′ to a temperature T2′ that is greater than T1′.

11. The process as claimed claim 9, wherein the temperature of the catalytic bed is increased by increasing the contact time, calculated as being the ratio of the volume, in liters, of catalyst to the total flow rate of said gaseous stream, in standard liters per second, at the inlet of the reactor.

12. The process as claimed claim 9, wherein step ii) is carried out at a temperature T3 and the temperature T2′ is less than the temperature T3 at which step ii) is carried out.

13. A process for the production of trifluoroethylene in a reactor provided with a catalytic bed comprising a catalyst, said process comprising:
a) a step of reacting chlorotrifluoroethylene with hydrogen, carried out in the presence of the catalyst, in the gas phase and at a temperature of the catalytic bed T3, in order to produce a stream comprising trifluoroethylene;
b) a step of regenerating the catalyst used in step a); and
c) repeating steps a) and b) using the catalyst regenerated in step b),
wherein step b) is carried out at a temperature of the catalytic bed T4 of from 90° C. to 300° C. and in the presence of hydrogen.

14. The process as claimed in claim 13, wherein the temperature difference ΔT between the temperature of the catalytic bed in step a) and the temperature of the catalytic bed in step b), ΔT=T3−T4, is, in absolute value, between 0 and 50.

15. The process as claimed claim 13, wherein step a) is preceded by a step of activating the catalyst.

16. The process as claimed claim 13, wherein it also comprises a step d) of heat treatment of said catalyst at a temperature T5 greater than T4.

17. The process as claimed claim 1 wherein said catalyst is a catalyst based on a metal from groups 8 to 10 of the periodic table.

18. A process for the production of trifluoroethylene in two reactors each comprising at least one catalytic bed comprising a catalyst, said process comprising:
in a first reactor, a step of reacting chlorotrifluoroethylene with hydrogen, carried out in the presence of the catalyst and in the gas phase in order to produce a stream comprising trifluoroethylene; and
in a second reactor, a step of heat treatment of said catalyst.

19. The process as claimed in claim 18, wherein said reaction step and said heat treatment step are carried out alternately in each of said two reactors.

20. The process as claimed claim 18, wherein said heat treatment step is a step of activating the catalyst comprising bringing it into contact with a gaseous stream comprising a reducing agent an inert gas or miniature thereof, or a step of regenerating the catalyst.
